(19) United States
(12) Patent Application Publication
(10) Pub. No.: US 2013/0130920 A1
(43) Pub. Date: May 23, 2013

(54) HIGH THROUGH-PUT ANALYSIS OF TRANSGENE BORDERS

(71) Applicant: Dow AgroSciences LLC, Indianapolis, IN (US)

(72) Inventors: Zehui Cao, Westfield, IN (US); Stephen Novak, Westfield, IN (US); Lakshmi Sastry-Dent, Indianapolis, IN (US); Ning Zhou, Zionsville, IN (US)

(73) Assignee: DOW AGROSCIENCES LLC, Indianapolis, IN (US)

(21) Appl. No.: 13/683,442

(22) Filed: Nov. 21, 2012

Related U.S. Application Data
(60) Provisional application No. 61/562,480, filed on Nov. 22, 2011.

(51) Int. Cl. C12Q 1/68

(52) U.S. Cl. CPC C12Q 1/686
USPC 506/2; 536/23.6; 435/6.12

(57) ABSTRACT

A method of analyzing, in chromosomal DNA having a transgene incorporated therein, a DNA flanking region derived from the chromosome which is adjacent to the transgene. Wherein, the DNA flanking region is characterized by isolation and digestion of genomic DNA with a restriction enzyme, ligation of a double stranded adapter to the isolated and digested genomic DNA, a primer extension reaction of the adapter ligated genomic DNA, and the isolation of the primer extension reaction product via a streptavidin-biotin interaction. The DNA flanking region is further characterized via subsequent PCR amplification reactions and DNA sequencing.

### CROSS REFERENCE TO RELATED APPLICATIONS

This application claims priority to U.S. Application No. 61/562,480, filed on Nov. 22, 2011, which is herein incorporated by reference in its entirety.

### TECHNICAL FIELD OF THE INVENTION

The subject invention relates generally to the fields of plant molecular biology and biochemistry. The subject invention concerns a modified Polymerase Chain Reaction (PCR) method for analyzing a transgene border and determining the chromosomal sequence which flanks the transgene.

### BACKGROUND OF THE INVENTION

Determining the genomic location and the chromosomal flanking sequence adjacent to an inserted transgene is technically challenging. Various methods have been developed to overcome the limitation of identifying the unknown DNA sequences which flank a known DNA sequence. However, these traditional PCR methods for the identification of genomic chromosomal sequences which flank a known transgene such as LM-PCR (also described as Genome Walking) and other methods including inverse PCR (i-PCR), thermal asymmetric interlaced PCR (TAIL-PCR), anchored PCR (a-PCR) and randomly primed PCR (rm-PCR) are hindered by low detection sensitivity (requiring large quantities of template DNA) or low specificity because of losses of DNA during preparation.

The development of a method which can improve detection sensitivity by purifying chromosomal DNA fragments which contain both the known and unknown DNA sequences can result in a sensitive method for detecting and characterizing unknown DNA regions which are located adjacent to a known DNA sequence. The development of the Linear Amplification Mediated Polymerase Chain Reaction (LAM PCR) method achieves these goals. see U.S. Pat. No. 6,514,706. The LAM PCR method is particularly suited to amplify and analyze DNA fragments, the sequence of which is only known in part.

Disclosed within this application is a modified LAM PCR method used to identify genomic chromosomal sequences which flank an inserted transgene. The disclosed method contains modifications to the traditional LAM PCR method that were developed to improve the accuracy, sensitivity, and reproducibility of the detection of the unknown chromosomal sequences which flank a known DNA sequence. This modified LAM PCR method can be deployed as a high throughput method to quickly and efficiently identify the genomic chromosomal sequences which flank a transgene. Further analysis of these sequences can be used to characterize the transgene insertion site for the identification of rearrangements, insertions and deletions which result from the integration of the transgene. In addition, analysis of the chromosomal flanking sequences can be used to identify the location of the transgene on the chromosome. Finally, the method can be broadly applied to determine an unknown DNA sequence which is adjacent to any known DNA sequence.

### BRIEF SUMMARY OF THE INVENTION

The subject invention provides a method for finding an unknown polynucleotide sequence adjacent to a known polynucleotide sequence in isolated plant DNA, which comprises: digesting the isolated plant DNA that contains a portion or all of the known polynucleotide sequence and an adjacent unknown polynucleotide sequence with one or more suitable restriction enzymes to produce a plurality of digested polynucleotide restriction fragments; ligating a double stranded adapter to the digested polynucleotide restriction fragments; synthesizing a complementary strand of the adapter ligated polynucleotide restriction fragments using an oligonucleotide primer sequence having an attachment chemistry bound to the 5′ end of the oligonucleotide primer sequence;

isolating the complementary strand by binding the attachment chemistry to a suitable isolation matrix;
performing a PCR amplification of the isolated complementary strand using a first PCR primer designed to bind to the known polynucleotide sequence and a second PCR primer designed to bind to the adapter sequence to produce a PCR amplicon; and,
sequencing the PCR amplicon to ascertain the sequence of the unknown polynucleotide sequence.

An embodiment of the subject invention, disclosed herein, is a method for the isolation and identification of transgene border sequences. An embodiment of the subject invention is a method which is readily applicable for high throughput analysis to determine the transgenic copy number and the chromosomal location of a genomic insertion site. In addition, the subject invention can be used for the simultaneous detection of multiple insertion sites within one reaction. The subject invention discloses a method which has improved sensitivity and specificity for the detection of unknown polynucleotide fragments which flank a known polynucleotide fragment. Moreover, the subject invention can be deployed to detect the unknown DNA sequences which are located adjacent to any target sequence, including viral sequences and insertional mutagenesis sites created via transposon mutagenesis or mutagenesis generated via T-strand integration.

An embodiment of the subject invention relates in part to transgenic event identification using such flanking, junction, and insert sequences. According to the subject invention, modified PCR analysis and DNA sequencing analysis methods using amplicons that span across inserted transgene DNA and its borders can be used to detect or identify commercialized transgenic plant varieties or lines derived from the proprietary transgenic plant lines.

The transgene border and adjacent chromosomal flanking sequences of the subject invention are diagnostic for a transgenic event. Based on these sequences, transgenic plant lines can be identified in different plant genotypes by analysis of the chromosomal flanking and transgene sequences. Thus, an embodiment of the subject invention describes a method that can be used to identify transgenic plant lines.

The chromosomal flanking sequences of the subject invention are especially useful in conjunction with plant breeding, to determine which progeny plants comprise a given event, after a parent plant comprising an event of interest is crossed with another plant line in an effort to impart one or more additional traits of interest in the progeny. An embodiment of the subject invention is the determination of the chromosomal flanking/junction sequences to benefit breeding programs as well as quality control, especially for commercialized transgenic plant lines.

Furthermore, the identification of chromosomal flanking sequences can be used to specifically identify the genomic location of each transgenic insert. This information can be used to develop molecular marker systems specific for each event. These molecular marker systems can be used for accelerated breeding strategies and to establish linkage data. An embodiment of the subject invention is the development of molecular marker systems for marker assisted breeding.

Still further, the chromosomal flanking sequence information can be used to study and characterize transgene integration processes, genomic integration site characteristics, event sorting, stability of transgenes and their flanking sequences, and gene expression (especially related to gene silencing, transgene methylation patterns, position effects, and potential expression-related elements such as MARS [matrix attachment regions], and the like).

The methods of this invention can be used to obtain and ascertain the sequence of the unknown polynucleotide from a transgenic organism. In any of the methods of this invention, the sample can be genomic DNA and the transgenic organism can be a transgenic plant. Transgenic plants analyzed by any of the methods of this invention can be selected from plants consisting of barley, corn, oat, sorghum, turf grass, sugarcane, wheat, alfalfa, banana, broccoli, bean, cabbage, canola, carrot, cassava, cauliflower, celery, citrus, cotton, a cucurbit, eucalyptus, flax, garlic, grape, onion, lettuce, pea, peanut, pepper, potato, poplar, pine, rye, rice, sunflower, safflower, soybean, strawberry, sugar beet, sweet potato, tobacco, tomato, ornamental, shrub, nut, millet, and pasture grass.

The methods of this invention can be used to obtain and ascertain the sequence of the unknown polynucleotide from a non-transgenic organism. In any of the methods of this invention, the sample can be genomic DNA and the non-transgenic organism can be a plant. Plants analyzed by any of the methods of this invention can be selected from plants consisting of barley, corn, oat, sorghum, turf grass, sugarcane, wheat, alfalfa, banana, broccoli, bean, cabbage, canola, carrot, cassava, cauliflower, celery, citrus, cotton, a cucurbit, eucalyptus, flax, garlic, grape, onion, lettuce, pea, peanut, pepper, potato, poplar, pine, rye, rice, sunflower, safflower, soybean, strawberry, sugar beet, sweet potato, tobacco, tomato, ornamental, shrub, nut, millet, and pasture grass. In any of the methods of the invention, the unknown polynucleotide sequence adjacent to a known polynucleotide sequence can be a native polynucleotide of agronomic interest.

### BRIEF DESCRIPTION OF THE SEQUENCES

SEQ ID NO:1 describes the adapter labeled “Adapter 5′.”

SEQ ID NO:2 describes the adapter labeled “Adapter 3′.”

SEQ ID NO:3 describes the primer labeled as “4468-3PA01-2Btn.”

SEQ ID NO:4 describes the primer labeled as “PAT-InvPriF: 5′.”

SEQ ID NO:5 describes the primer labeled as “lmAdp-Pr.”

SEQ ID NO:6 describes the 106685[1]-001 chromosomal flanking sequence, adapter, and cloning vector which was sequenced using the method.

SEQ ID NO:7 describes only the chromosomal flanking sequence of event 106685[1]-001 adjacent to the inserted transgene.

SEQ ID NO:8 describes the 106685[1]-010 chromosomal flanking sequence, the 3′ flank of the inserted transgene, adapter, and cloning vector which was sequenced using the method.

SEQ ID NO:9 describes only the chromosomal flanking sequence of event 106685[1]-010 adjacent to the inserted transgene.

SEQ ID NO:10 describes the 106685[1]-013 chromosomal flanking sequence adapter, and cloning vector which was sequenced using the method.

SEQ ID NO:11 describes only the chromosomal flanking sequence of event 106685[1]-013 adjacent to the inserted transgene.

SEQ ID NO:12 describes the 106685[1]-035 chromosomal flanking sequence, adapter, and cloning vector which was sequenced using the method.

SEQ ID NO:13 describes only the chromosomal flanking sequence of event 106685[1]-035 adjacent to the inserted transgene.

### DETAILED DESCRIPTION OF THE INVENTION

The polymerase chain reaction (PCR) is a commonly employed molecular biology method. The method is performed by denaturing double-stranded template DNA, annealing oligonucleotide primers to the DNA template, and extension of a DNA strand via a DNA polymerase. The oligonucleotide primers are designed to anneal to opposite strands of the DNA and positioned so that the DNA strand produced by the DNA polymerase serves as a template strand for the other primer. Each cycle is repeated, resulting in the exponential amplification of a DNA fragment. (Mullis et al., U.S. Pat. Nos. 4,683,195, 4,683,202, and 4,800,159). The use of PCR by those skilled in the art is fundamental for amplifying and isolating DNA fragments for subsequent analysis.

Isolation and analysis of DNA templates via the polymerase chain reaction (PCR) requires knowledge of the flanking DNA sequences. Unfortunately, this requirement limits PCR amplification to regions of known DNA sequence. The use of PCR methodologies to identify the location of a transgene location within a genome is hindered by the random insertion of the transgene into an unknown chromosomal location within the genome of an organism. Methods to identify unknown DNA sequences which are located adjacent to a known DNA sequence are necessary for the identification of a transgene location within the chromosome of an organism. In addition such methods can be used to identify novel gene sequences to identify new traits, to determine the genomic location of a transposon or viral sequence which has been inserted into the genome of an organism, or to identify the chromosomal location of polynucleotide sequences inserted into the genome via insertion mutagenesis.

Various methods have been developed to overcome the limitation of the unknown DNA sequences which flank a known DNA sequence. A Ligation Mediated PCR (LM PCR) method wherein a genomic library is generated and adapters are annealed to DNA fragments for PCR amplification is marketed as the GENOME WALKER UNIVERSAL KIT™ (see U.S. Pat. No. 5,565,340, and U.S. Pat. No. 5,759,822). Another method commonly used is the inverse PCR reaction (see Silver and Keerikatte (1989), J. Virol., 63:1924-1928), wherein DNA is digested with a restriction enzyme and self ligated resulting in a contiguous circle. PCR amplification using oligonucleotide primers which bind to known sequences results in amplification and elucidation of the unknown flanking sequences. Unfortunately these methods are inefficient and time consuming. These and other traditional PCR methods (including thermal asymmetric interlaced PCR [TAIL-PCR], anchored PCR [a-PCR] and randomly primed PCR [rm-PCR]) are hindered by low detection sensitivity (requiring large quantities of template DNA) or low specificity because of losses of DNA during preparation.

The development of a method which can improve detection sensitivity by purifying chromosomal DNA fragments which contain both the known and unknown DNA sequences can result in a sensitive method for detecting and characterizing unknown DNA regions which are located adjacent to a known DNA sequence. The development of the LAM PCR method achieves these goals. The Linear Amplification Mediated Polymerase Chain Reaction (LAM PCR) method is a modified PCR method that is used for analyzing unknown chromosomal flanking sequences located adjacent to a known DNA sequence. The LAM PCR method can be used to identify and/or sequence an unknown DNA or RNA sequence flanking a known DNA or RNA region.

The LAM PCR method consists of the following steps. A primer extension reaction is performed using a chromosomal DNA as a template and an oligonucleotide primer which binds to a known DNA sequence within the chromosomal DNA. The oligonucleotide primer is complementary to a long terminal repeat (LTR) sequence, which is a sequence characteristic of a retrovirus, and labeled with biotin at the end of the oligonucleotide primer. The single-stranded DNA product of the linear PCR is bound to magnetic beads having immobilized streptavidin. This step serves to isolate the single-stranded amplified DNA fragment containing the known LTR sequence and an unknown sequence derived from the chromosome. The single-stranded DNA is converted into a double-stranded DNA by synthesizing the complementary strand. The double-stranded DNA is cleaved with a restriction enzyme that recognizes a sequence and cleaves the double-stranded DNA at the sequence. A double-stranded DNA called a linker cassette is ligated to the terminus. Subsequent PCR reactions are conducted using the thus obtained ligation product as a template as well as a primer complementary to the LTR and a primer complementary to the linker cassette. A DNA fragment that contains the LTR and chromosome DNA flanking sequence adjacent to the LTR is amplified. As a result the previously unknown retrovirus integration site can be determined.

The LAM PCR method is currently considered to be an effective system for analyzing unknown DNA sequences adjacent to a known DNA sequence. However, modifications and improvements to the LAM PCR method have been described in the art. see U.S. Pat. App. US2007/0037139 and Harkey et al., (2007) Stem Cells Dev., June; 16(3): 381-392.

The LAM PCR method was modified in U.S. Pat. App. US2007/0037139 to improve the detection of a biological sample having a retrovirus integrated at various sites. The reaction conditions of the traditional LAM PCR method produced results that did not reflect the actual state of clones existing in the cell population of the sample. A modification was developed in which more integration fragments were PCR amplified without being biased toward a fragment amplified from a specific clone. The modification to the LAM PCR method allowed researchers to determine the extent of cells having an integrated gene in the population and to determine the ratio of a specific cell in the population.

In addition, Harkey et al., (2007) describe an optimized, multiarm, high throughput modification of the LAM PCR method wherein the detection capacity was improved 90% with exhaustive sampling. The modified protocol facilitated accurate estimates of the total pool size, thus providing a rapid, cost-effective approach for generating large insertion-site data of preferred genomic locations for vector integration.

The subject invention describes another modification wherein, the traditional LAM-PCR method which requires the steps of completion of a primer extension reaction, generation of a double stranded DNA fragment and, the subsequent digestion of the double stranded DNA fragment with restriction enzymes are not required. Rather the subject invention describes an initial restriction enzyme digestion of gDNA, the ligation of a double stranded adapter to the gDNA fragment and a primer extension reaction. In addition to other modifications of the method.

As used herein, the terms “comprises,” “comprising,” “includes,” “including,” “has,” “having,” “contains,” or “containing,” or any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The terms “nucleic acid,” “polynucleotide,” “polynucleotide sequence,” and “nucleotide sequence” are used to refer to a polymer of nucleotides (A,C,T,U,G, etc. or naturally occurring or artificial nucleotide analogues), e.g., DNA or RNA, or a representation thereof, e.g., a character string, etc, depending on the relevant context. The terms “nucleic acid” and “polynucleotide” are used interchangeably herein; these terms are used in reference to DNA, RNA, or other novel nucleic acid molecules of the invention, unless otherwise stated or clearly contradicted by context. A given polynucleotide or complementary polynucleotide can be determined from any specified nucleotide sequence. A nucleic acid may be in single- or double-stranded form.

The term “isolated,” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

The term “plant,” includes plants and plant parts including but not limited to plant cells and plant tissues such as leaves, stems, roots, flowers, pollen, and seeds. The class of plants that can be used in the present invention is generally as broad as the class of higher and lower plants amenable to mutagenesis including angiosperms (monocotyledonous and dicotyledonous plants), gymnosperms, ferns and multicellular algae.

The term “promoter,” typically refers to a DNA sequence which directs the transcription of a structural gene to produce RNA. Typically, a promoter is located in a region 500 base pairs upstream of a gene, proximal to the transcription start site. If a promoter is an inducible promoter, then the rate of transcription increases or decreases in response to an exogenous or endogenous inducing agent. In contrast, the rate of transcription is regulated to a lesser degree by an inducing agent if the promoter is a constitutive promoter.

The term “transgenic plant,” refers to a plant or progeny thereof derived from a transformed plant cell or protoplast, wherein the plant DNA contains an introduced exogenous DNA molecule not originally present in a native, non-transgenic plant of the same.

The term “vector,” as used herein refers to any recombinant polynucleotide construct that may be used for the purpose of transformation, i.e., the introduction of heterologous DNA into a host cell.

The term “complementary strand,” describes nucleic acid sequences or molecules in which each base in one molecule is paired with its complementary base in the other strand, to form a stable helical double strand molecule. The individual strands are termed complementary strands.

The term “oligonucleotide primer,” is a sequence of linear oligonucleotides of about ten to about fifty nucleotides in length that are complementary to nucleotide sequences 5′ or 3′ to be amplified. A pair of oligonucleotide primers, in which one of the primers is complementary to a nucleotide sequence 5′ of the polynucleotide fragment to be amplified while the other primer of the pair is complementary to a nucleotide sequence located 3′ polynucleotide fragment to be amplified can be used to amplify a polynucleotide sequence. One skilled in the art understands that a pair of oligonucleotide primers means two oligonucleotides complementary to opposite strands of nucleic acid and flanking the polynucleotide sequence to be amplified.

The term “adapter,” describes a short, oligonucleotide polynucleotide segment that can be joined to a polynucleotide molecule at either a blunt end or cohesive end. Adapters may contain restriction enzyme recognition sequences within the polynucleotide fragment. The size of the adapter can vary from about ten to about one-hundred and fifty nucleotides in length. Adapters can either be single stranded or double stranded.

The term “isolated complementary strand,” refers to a polynucleotide fragment which comprises an adapter joined to a second DNA fragment that contains a portion or all of the known polynucleotide sequence and an adjacent unknown polynucleotide sequence via a ligation reaction. The “isolated complementary strand” is flanked by an adapter on one end and a known polynucleotide sequence on the other end.

A ligation reaction is completed by an enzyme, generally referred to as a ligase that catalyzes the formation of a phosphodiester bond between adjacent 3′-OH and 5′-P termini in DNA.

Isolation of a plant DNA can be accomplished by methods known in the art. Generally, the isolation of a plant DNA results in obtaining purified plant DNA which is free of lipids, proteins and other cellular debris. Preferred plant DNA isolation methods include: lysis, heating, alcohol precipitation, salt precipitation, organic extraction, solid phase extraction, silica gel membrane extraction, CsCl gradient purification, and any combinations thereof. A more preferred plant DNA isolation method is the silica-gel-membrane technology marketed as the DNeasy kit (Qiagen, Valencia, Calif.) or the Cetyltrimethylammonium Bromide (CTAB) DNA isolation protocol.

Restriction enzyme digestions, also referenced as restriction endonuclease digestions, are performed when a nuclease enzyme is used to cleave the polynucleotide sequences. There are numerous restriction enzymes available to those skilled in the art. As described at www.neb.com/nebecomm/tech_reference/restriction_enzymes/overview.asp, four classifications are used to characterize restriction enzymes. These classifications are made on the basis of subunit composition, cleavage position, sequence specificity and cofactor requirements.

Type I enzymes randomly cut DNA at locations which are a distance from the recognition/binding sequence (>1,000 bp away). The recognition sites which are bound by a Type I enzyme are asymmetrical. As a result these enzymes are not used for gene cloning because these enzymes do not produce discrete restriction fragments or distinct gel-banding patterns. Type I enzymes are multifunctional and the different subunits which comprise a Type I restriction enzyme are responsible for different activities (i.e. subunit HsdR encodes restriction, subunit HsdM encodes methylation of DNA, and subunit HsdS encodes specificity of the recognition sequence).

Type II enzymes digest DNA at positions located within close proximity of the recognition sequences. These enzymes function as a dimer, wherein a subunit binds to the sense strand and a second copy of the subunit binds to the antisense strand at a palindromic sequence which is typically between 4-8 nucleotides in length. The Type II dimer that binds to the DNA can be either a homodimer which bind to symmetric DNA sequences, or a heterodimer which binds to asymmetric DNA sequences. The enzymes can recognize either continuous sequences or discontinuous sequences. Type II enzymes are commercially available and commonly used for DNA analysis and gene cloning. Widespread usage of these enzymes is a result of distinct restriction fragments which are produced and can be resolved on an agarose gel.

Type II enzymes are a collection of unrelated proteins which are highly divergent in amino acid sequence similarity. Type II enzymes have been divided into subcategories which are labeled using a letter suffix. Type IIB restriction enzymes are multimers that contain more than one subunit. These enzymes cut both sides of the recognition sequence, thereby resulting in removal of the recognition sequence. Type IIE and Type IIF restriction enzymes cleave DNA following interaction with two copies of their recognition sequence. Type IIG restriction enzymes are comprised of a single subunit. The N-terminal portion of the enzyme possesses a DNA cleavage domain and DNA modification domain. The C-terminal portion of the enzyme possesses a DNA sequence binding domain. These enzymes cleave outside of their recognition sequence. Type IIM restriction enzymes recognize and cut methylated DNA. Type IIS restriction enzymes function as a dimer and cleave DNA at a location which is outside of the non-palindromic asymmetric recognition sites. These enzymes are comprised of two distinct domains, one for DNA binding and the other for DNA cleavage.

Type III enzymes are combination restriction-and-modification enzymes. These enzymes recognize two separate non-palindromic sequences and cleave outside of their recognition sequences. Type III enzymes require two recognition sequences in opposite orientations within the same DNA molecule to accomplish cleavage.

Type IV enzymes recognize methylated DNA. Examples include the McrBC and Mrr systems of E. coli.

Other methods are known in the art for cleaving polynucleotides and can be used in place of digesting the polynucleotide with a restriction enzyme, any of the group consisting of: lysis, a sequence-specific cleavage agent, non-sequence specific cleavage agent, sonication, shear-stress, French press, UV radiation, ionizing radiation, and DNase. In addition, to the restriction enzymes described above, homing endonucleases or Flap endonucleases or any combination of these enzymes could be used to digest the isolated DNA. A preferred method for digesting isolated plant DNA is the use of a TypeII restriction enzyme which is known to cut outside of the transgene sequence being transformed into the plant. Another preferred method for digesting isolated plant DNA is the use of a TypeII restriction enzyme which is known to cut at a site which is in close proximity of the end of the transgene sequence.

Primer extension reactions are used to produce a DNA or RNA strand which contains a known polynucleotide sequence and an unknown adjacent polynucleotide sequence. Primer extension methodologies result in the production of a complementary strand of DNA or RNA which contains the unknown polynucleotide sequence. The complementary strand of DNA or RNA is produced by a polymerase which extends along a template strand of DNA or RNA after complexing with an oligonucleotide primer which has bound to the known template strand of DNA or RNA. The oligonucleotide primer is designed to specifically bind to the known DNA or RNA sequence within the template strand of DNA or RNA. Numerous types of polymerase are commercially available for the extension reaction; T4 polymerase, TAQ polymerase, PFU polymerase, or Reverse Transcriptase are a few non-limiting examples of commonly used polymerases. Each polymerase has special buffer requirements and function at a specific temperature for optimal reaction conditions. A preferred primer extension reaction is the use of the TAQ polymerase marketed as the Platinum Taq kit.

Attachment chemistries attached to an isolation matrix such, as magnetic bead-based systems, are used to isolate the DNA produce by the primer extension reaction. The DNA strand which is produced by the primer amplification reaction can be purified from genomic DNA via a streptavidin—biotin interaction. Biotinylation is widely used to enable isolation, separation, concentration and further downstream processing and analysis of biomolecules (for example, methods described in U.S. Pat. No. 5,948,624, U.S. Pat. No. 5,972,693, and U.S. Pat. No. 5,512,439). There are a variety of commercially available biotinylation reagents that target different functional groups like primary amines, sulfhydryls, carboxyls, carbohydrates, tyrosine and histidine side chains and cyianidine and cytosine bases. The use of short, sequence-specific oligonucleotide primers functionalized with biotin (or the equivalent, e.g. digoxigenin) and magnetic beads to separate specific DNA sequences from the genome for subsequent analysis have multiple uses. Isolation using the bead-based method allows for enrichment of a population of DNA for a particular sequence, allowing subsequent analysis to be carried out that could not be done in the presence of the entire genomic complement of DNA. Such bead-based methods are suited for high throughput automation.

Although the biotin—streptavidin interaction is the best described binding pair, other molecules which have a strong affinity for one another are known. Attachment chemistries that can be included into a oligonucleotide primer include: ACRYDITE™ an attachment chemistry based on an acrylic phosphoramidite that can be added to oligonucleotides as a 5′-modification, and covalently reacts with thiol-modified surfaces; Alkyne modifications which react with azide labeled functional groups to form stable bonds through the azide alkyne Huisgen cycloaddition reaction (also referenced as the Click reaction); and, Thiol modifications which can couple and interact with high affinity to a corresponding ligand or surface (such as a gold surface). These molecules can be used for purification or enrichment of DNA sequences. Wherein, a primer is labeled with a first molecule and the second molecule is bound to a matrix which can immobilize the first molecule (e.g. magnetic beads). A DNA strand produced from the primer labeled with the first molecule can be isolated by running the DNA over a column containing the immobilized matrix (e.g. magnetic beads) labeled with the second molecule. As a result of the affinity for the second molecule, the amplified DNA sequences containing the primer labeled with the first molecule are isolated. Preferred attachment chemistries include acrylic—thiol interactions, alkyne—azide interactions, and thiol—ligand interactions. A more preferred attachment chemistry is the streptavidin—biotin interaction.

As used herein, the term isolation matrix refers to a surface to which a molecule of any sort may be attached. Preferably, an isolation matrix is an insoluble material to which a molecule may be attached so that said molecule may be readily separated from other components in a reaction. Preferred isolation matrices may include, but is not limited to, a filter, a chromatography resin, a bead, a magnetic particle, or compositions that comprise glass, plastic, metal, one or more polymers and combinations thereof. A more preferred isolation matrix is the magnetic bead-based system.

Adapters can be ligated to double stranded genomic DNA via a ligase. Commercially supplied ligases are widely available for joining double stranded DNA fragments. Preferred double stranded ligases are commercially available and marketed as T4 Ligase (New England Biolabs; Ipswich, Mass. or Roche Biosciences; Indianapolis, Ind.), Taq Ligase (New England Biolabs; Ipswich, Mass.), and E. coli DNA Ligase (New England Biolabs; Ipswich, Mass.). A more preferred double stranded DNA ligase is the Quick Ligation kit from New England Biolabs (Ipswich, Mass.).

As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction. Three NGS platforms are commercially available, the Genome Sequencer FLX from 454 Life Sciences/Roche, the Illumina Genome Analyser from Solexa and Applied Biosystems' SOLiD (acronym for: ‘Sequencing by Oligo Ligation and Detection’). In addition, there are two single molecule sequencing methods that are currently being developed. These include the true Single Molecule Sequencing (tSMS) from Helicos Bioscience and the Single Molecule Real Time sequencing (SMRT) from Pacific Biosciences.

The Genome Sequencher FLX which is marketed by 454 Life Sciences/Roche is a long read NGS, which uses emulsion PCR and pyrosequencing to generare sequencing reads. DNA fragments of 300-800 bp or libraries containing fragments of 3-20 kbp can be used. The reactions can produce over a million reads of about 250 to 400 bases per run for a total yield of 250 to 400 megabases. This technology produces the longest reads but the total sequence output per run is low compared to other NGS technologies.

The Illumina Genome Analyser which is marketed by Solexa is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The Sequencing by Oligo Ligation and Detection (SOLiD) system marketed by Applied Biosystems is a short read technology. This NGS technology uses fragmented double stranded DNA that are up to 10 kbp in length. The system uses sequencing by ligation of dye-labelled oligonucleotide primers and emulsion PCR to generate one billion short reads that result in a total sequence output of up to 30 gigabases per run.

tSMS of Helicos Bioscience and SMRT of Pacific Biosciences apply a different approach which uses single DNA molecules for the sequence reactions. The tSMS Helicos system produces up to 800 million short reads that result in 21 gigabases per run. These reactions are completed using fluorescent dye-labelled virtual terminator nucleotides that are described as a ‘sequencing by synthesis’ approach.

The SMRT Next Generation Sequencing system marketed by Pacific Biosciences uses a real time sequencing by synthesis. This technology can produce reads of up to 1000 bp in length as a result of not being limited by reversible terminators. Raw read throughput that is equivalent to one-fold coverage of a diploid human genome can be produced per day using this technology.

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

The disclosure of each reference set forth herein is incorporated herein by reference in its entirety.

### EXAMPLES

The following examples describe a protocol which was developed to isolate and identify the genomic flanking sequences of a transgene insert. In addition, the protocol can be used to determine the transgene copy number and the genomic location of a transgene for a transgenic event.

### Example 1

A plasmid containing a polynucleotide of interest expression cassette and a selectable marker gene expression cassette was used to transform Zea mays cv Hi-II plant tissue via the Biorad gene gun. Frame, et al., Production of transgenic maize from bombarded Type II callus: effect of gold particle size and callus morphology on transformation efficiency. In Vitro Cell. Dev. Biol-Plant. 36:21-29). The protocol was modified: media components, selection agents and timing were optimized to improve the efficiency of the transformation process. An Fsp I linearized fragment of the plasmid was used for the transformation. The resulting transformations produced transgenic maize plants which contained a polynucleotide of interest expression cassette which was linked to the plant selectable marker gene expression cassette. The following transgenic events were produced; 106685[1]-001, 106685[1]-010, 106685[1]-013, and 106685[1]-035. These events were used to exemplify the detection method.

### Example 2

Genomic DNA (gDNA) was isolated from different maize events and untransformed maize controls. Several methods were employed to isolate the gDNA, such as the DNeasy kit (Qiagen, Valencia, Calif.) or the traditional Cetyl trimethylammonium bromide (CTAB) DNA isolation protocol. The DNA concentrations were determined using a Nanodrop (Thermo Scientific, Wilmington, Del.). A total of 250 ng of gDNA was digested with a restriction enzyme. Restriction enzymes that bind to degenerate recognition sequences, such as AflIII (recognizes the sequence ACRYGT), BanI (recognizes the sequence GGYRCC), BstYI (recognizes the sequence RGATCY), and StyI (recognizes the sequence CCWWGG), were used to achieve higher levels of cutting frequency which is comparable to four or six base pair cutting restriction enzymes. The resulting digestion fragments were of medium sized lengths, ˜1,000 bp. The digestion reaction was further purified using the MinElute Reaction Cleanup Kit (Qiagen, Valencia, Calif.).

### Example 3

A double stranded adapter (SEQ ID NO:1 Adapter 5′:5′-Phosphorylation-GYRCAGCGGATCGTCT-Inverted dT-3′ which was annealed with SEQ ID NO:2 Adapter 3′: 5′-GTCCGACCGTCAGAGAATCCAATAGACGATCCGCT-3′) was ligated to the digested gDNA. The ligation reaction was completed using the Quick Ligation kit (New England Biolabs, Ipswich, Mass.) per manufacturer's instructions. The ligation reaction was incubated at 25° C. for 30 minutes and then the reaction was stopped by heating the cocktail to 80° C. for 15 minutes. The reaction was stored indefinitely at 4° C.

### Example 4

Primer extension reactions using the adapter ligated gDNA were completed. A gene specific primer was synthesized by Integrated DNA Technologies Inc. (Coralville, Iowa) and used for the reaction (SEQ ID NO:34468-3PA01-2Btn: 5′-\Dual Biotin\-GGACAGAGCCACAAACACCACAAGA-3′). The Platinum Taq kit (Invitrogen, Carlsbad, Calif.) was used to synthesize a DNA strand via primer extension. The following reagents: 1 μL, 10× platinum TAQ buffer; 0.2 μL, 10 mM dNTP; 0.1 μL, 10 μM 4468-3PA01-2Btn; 0.05 μL Patinum TAQ; 6.7 μL H₂O; 2 μL of adaptor-ligated product were mixed in a tube. Amplification was completed using the following reaction conditions: 1) 94° C. 2.5 minutes; 2) 94° C. 30 seconds; 3) 69° C. 6 minutes; 4) repeat steps 2-3 5 times; 5) 4° C. hold.

### Example 5

A capture reaction was completed with 2.0 μL of DYNABEADS® M-270 streptavidin magnetic beads (Invitrogen, Carlsbad, Calif.). The beads were washed on a magnet with PBST buffer (phosphate buffered saline and tween 20) three times. The beads were resuspended in 10 μL of PBS (phosphate buffered saline) containing 0.1% Tween 20 and 0.5 μl of 10 mg/ml of sheared salmon sperm DNA, the beads were mixed vigorously in the PBS solution. The PBS solution was added to the primer extension reaction at a 1:1 concentration, 20 μL of beads were mixed with 20 μL of primer extension reaction. The resulting solution was incubated for thirty minutes with gentle pipetting at room temperature. The beads containing the primer extension reaction were then washed over a magnet three times with 50 μl of washing buffer (10 mM Tris (pH 8.0)-1 mM EDTA containing 0.1% Tween 20) and then one time with 50 μA of dd-H₂0. All of the wash solutions were removed from the beads.

### Example 6

PCR reactions were completed using the Takara EX TAQ HS PCR kit (Millipore, Billerica, Ma). The following primers were used to amplify the event and flanking sequence: Transgene specific primer, (SEQ ID NO:4 PAT-InvPriF: 5′-CGCTTACGATTGGACAGTTGAGAGTACTG-3′) and Adaptor primer, (SEQ ID NO:5 lmAdp-Pri: 5′-GTCCGACCGTCAGAGAATCCAAT-3′). The following reagents were used in the PCR reaction: 5 μL, 10× EX-TAQ HS buffer; 4 μL, 2.5 mM dNTP, 1 μL, 10 μM transgene specific primer; 1 μL, 10 μM adapter specific primer; 0.25 μL EX-Taq HS polymerase; and, 38.75 μL, H₂0. The cocktail was added to the washed beads from the ligation reaction, resuspended well and amplified using the following conditions in Table 1.

TABLE 1
PCR amplification conditions

| | |
|---|---|
| 1 cycle | 94° C., 2 min |
| 1 cycle | 98° C., 10 sec |
| | 68° C., 6 min |
| 30 cycles | 98° C., 10 sec |
| | 68° C., 4 min |
| 1 cycle | 72° C., 4 min |
| 1 cycle | 4° C., ∞ |

### Example 7

The resulting PCR products were cloned into plasmid pCR2.1 (Invitrogen, Carlsbad, Calif.). Colonies were isolated and the pCR2.1 plasmid was confirmed to contain a PCR amplicon. The vectors were sequenced using M13 Forward and M13 Reverse primers. The sequencing results were expected to contain the nucleotide sequence of the maize 3′ genomic flanking sequence in addition to the genetic elements present from the plasmid. The 3′ transgene insert and maize genomic flanking sequences from events 106685[1]-001, 106685[1]-010, 106685[1]-013, and 106685[1]-035 were isolated and identified using the technique described above.

The characterization of the genomic insertions indicated that event 106685[1]-001 contains one copy of the transgene. The sequencing data of the chromosomal flanking region (SEQ ID NO:6) indicated that one copy of the transgene inserted into a unique location of the Zea mays genome. The site of insertion of the 3′ end was mapped to maize chromosome 10:140567138 . . . 140568099, (SEQ ID NO:7).

The characterization of the genomic insertions indicated that event 106685[1]-010 contains one copy of the transgene. The sequencing data of the chromosomal flanking region (SEQ ID NO:8) indicated that one copy of the transgene inserted into a unique location of the Zea mays genome. The site of insertion of the 3′ end was mapped to maize chromosome 8:161488669 . . . 161489160 (SEQ ID NO:9).

The characterization of the genomic insertions indicated that event 106685[1]-013 contains one copy of the transgene. The sequencing data of the chromosomal flanking region (SEQ ID NO:10) indicated that one copy of the transgene inserted into a unique location of the Zea mays genome. The site of insertion of the 3′ end was mapped to maize chromosome 1:43455257 . . . 43456230 (SEQ ID NO:11).

The characterization of the genomic insertions indicated that event 106685[1]-035 contains one copy of the transgene. The sequencing data of the chromosomal flanking region (SEQ ID NO:12) indicated that one copy of the transgene inserted into a unique location of the Zea mays genome. The site of insertion of the 3′ end was mapped to maize chromosome 1:2075090 . . . 2076004 (SEQ ID NO:13).

What is claimed is:

1. A method for finding an unknown polynucleotide sequence adjacent to a known polynucleotide sequence in isolated plant DNA, which comprises:
digesting the isolated plant DNA that contains a portion or all of the known polynucleotide sequence and an adjacent unknown polynucleotide sequence with one or more suitable restriction enzymes to produce a plurality of digested polynucleotide restriction fragments;
ligating a double stranded adapter to the digested polynucleotide restriction fragments;
synthesizing a complementary strand of the adapter ligated polynucleotide restriction fragments using an oligonucleotide primer sequence having an attachment chemistry bound to the 5′ end of the oligonucleotide primer sequence;
isolating the complementary strand by binding the attachment chemistry to a suitable isolation matrix;
performing a PCR amplification of the isolated complementary strand using a first PCR primer designed to bind to the known polynucleotide sequence and a second PCR primer designed to bind to the adapter sequence to produce a PCR amplicon; and
sequencing the PCR amplicon to ascertain the sequence of the unknown polynucleotide sequence.

2. The method of claim 1, wherein the plant DNA is plant genomic DNA.

3. The method of claim 1, wherein the unknown polynucleotide sequence is a transgene border.

4. The method of claim 1, wherein the unknown polynucleotide sequence is a chromosomal sequence which flanks a known polynucleotide sequence.

5. The method of claim 1, wherein the unknown polynucleotide sequence is an endogenous gene sequence which encodes a trait.

6. The method of claim 1, wherein the known polynucleotide sequence is a known polynucleotide viral sequence.

7. The method of claim 1 wherein the known polynucleotide sequence is a known polynucleotide transgene sequence.

8. The method of claim 1, wherein the known polynucleotide sequence is a known polynucleotide transposon sequence.

9. The method of claim 1, wherein the known polynucleotide sequence is a known polynucleotide gene sequence that encodes a trait.

10. The method of claim 1, wherein the method is used to identify the chromosomal location of a known polynucleotide sequence inserted into the isolated plant DNA via insertion mutagenesis.

11. The method of claim 10, wherein said insertion mutagenesis is selected from the group consisting of transposon mutagenesis, or T-strand integration mutagenesis.

12. The method of claim 1, wherein the method is used to characterize an unknown polynucleotide sequence consisting of a chromosomal sequence which flanks a known polynucleotide sequence.

13. The method of claim 12, wherein said characterization of a transgene insertion site identifies polynucleotide sequence consisting of rearrangements, insertions, deletions, or inversions within the unknown polynucleotide sequence consisting of a chromosomal sequence.

14. The method of claim 1, wherein the method is used in a high throughput protocol.

15. The method of claim 1, wherein the method is used to determine transgene copy number.

16. The method of claim 1, wherein the method is used to identify transgenic plant lines.

17. The method of claim 1, wherein the method is used to develop molecular marker systems.

18. The molecular marker systems of claim 17 that are used to accelerate breeding strategies.


SEQUENCE LISTING (13 sequences)

SEQ ID NO: 1 - DNA, 16 bp, Artificial Sequence
  Adapter 5′
gyrcagcggatcgtct

SEQ ID NO: 2 - DNA, 35 bp, Artificial Sequence
  Adapter 3′
gtccgaccgtcagagaatccaatagacgatccgct

SEQ ID NO: 3 - DNA, 25 bp, Artificial Sequence
  4468-3PA01-2Btn
ggacagagccacaaacaccacaaga

SEQ ID NO: 4 - DNA, 29 bp, Artificial Sequence
  PAT-InvPriF
cgcttacgattggacagttgagagtactg

SEQ ID NO: 5 - DNA, 23 bp, Artificial Sequence
  lmAdp-Pri
gtccgaccgtcagagaatccaat

SEQ ID NO: 6 - DNA, 1247 bp, Artificial Sequence
  106685[1]-001 chromosomal flanking sequence, adapter, and cloning vector sequence which was produced by the method
nnnnnnnnnnnnntgcntgctcgagcggccgccagtgtgatggatatctgcagaattcgcccttgtccgaccgtcagagaatccaatgacgatccgctgcgcctgctacctggttgtccgccggggagacgatttccggccaccgaaaggtacgtcgcatagccatgaatccagttcgtgaaataagaaatcacatggatacttcttcgcgatgcgagactgttgggccatggccatacgcgaaacgtggagccgtacctccattttctcggcgatggtgttcatgcagggagggtcctcgtgagggccgattaactgcctgttaattgaactggcggaagccggccgtagtcgaaaaggggcggaagcgcggtcagcacggacagatcatcgatccgacactgaagcgaattagcggttactaataagcagcggcatttctcacttgtaaaccatgagggtggcgacgtggagggagttgatgtccaggagccccgtgttcttgtctgccatgtcctcgaagacttccttgcagatcctctcgacgtcagacttgcctgcaaagcgaacacacagcgttaggatggactggacgtggacgaactcgatgcgtgagcggatcagagaagagaaacgaaaaggttgcagaaatggcggcgatatcgagagatgcgtgcgtgtgtgcaaggcagggagacgtggtctaccgcgtgctctgtcccacgcgcggccgaagaagctagacttcttggacgaggacatcgttggctccctcgagatctggattgatggcaactagccaactacacgacgagttggagaagggagggtggtcggccggtggcattatcaatgtggcggaatagctttttcaagtgtctgacgaagaagtcgcttcgcttgcttgcagggaanggcagaacagaatcagaaggctgccgactggctaatggcgtctctgttctgtgcagcagcctgtgcttatctcccttccngccggncctagcgccngtacttctttccttgctccgtcagcagttgagcacctanaacnagcccnggnatgccnttccttannnnttttgnctganannncnnngtnnngntcnaantnggactagtcntccnccgctggaangggnnncnatcagcnannnnnnnnnttnnttnnnnntnnntctnnnnnnncnanaannnnnnnnnnnnnnnnnnnnntnnnttnnnnnnnnnnnncnntnnnnnnnann

SEQ ID NO: 7 - DNA, 1160 bp, Artificial Sequence
  106685[1]-001 chromosomal flanking sequence adjacent to an inserted transgene
gacgatccgctgcgcctgctacctggttgtccgccggggagacgatttccggccaccgaaaggtacgtcgcatagccatgaatccagttcgtgaaataagaaatcacatggatacttcttcgcgatgcgagactgttgggccatggccatacgcgaaacgtggagccgtacctccattttctcggcgatggtgttcatgcagggagggtcctcgtgagggccgattaactgcctgttaattgaactggcggaagccggccgtagtcgaaaaggggcggaagcgcggtcagcacggacagatcatcgatccgacactgaagcgaattagcggttactaataagcagcggcatttctcacttgtaaaccatgagggtggcgacgtggagggagttgatgtccaggagccccgtgttcttgtctgccatgtcctcgaagacttccttgcagatcctctcgacgtcagacttgcctgcaaagcgaacacacagcgttaggatggactggacgtggacgaactcgatgcgtgagcggatcagagaagagaaacgaaaaggttgcagaaatggcggcgatatcgagagatgcgtgcgtgtgtgcaaggcagggagacgtggtctaccgcgtgctctgtcccacgcgcggccgaagaagctagacttcttggacgaggacatcgttggctccctcgagatctggattgatggcaactagccaactacacgacgagttggagaagggagggtggtcggccggtggcattatcaatgtggcggaatagctttttcaagtgtctgacgaagaagtcgcttcgcttgcttgcagggaanggcagaacagaatcagaaggctgccgactggctaatggcgtctctgttctgtgcagcagcctgtgcttatctcccttccngccggncctagcgccngtacttctttccttgctccgtcagcagttgagcacctanaacnagcccnggnatgccnttccttannnnttttgnctganannncnnngtnnngntcnaantnggactagtcntccnccgctggaangggnnncnatcagcnannnnnnnnnttnnttnnnnntnnntctnnnnnnncnanaannnnnnnnnnnnnnnnnnnnntnnnttnnnnnnnnnnnncnntnnnnnnnann

SEQ ID NO: 8 - DNA, 1226 bp, Artificial Sequence
  106685[1]-010 chromosomal flanking sequence, adapter, 3′ transgene,  and cloning vector sequence which was produced by the method
nnnnnnnnnnnnnnnnnctcggntccnctagtaacggccgccagtgtgctggaattcgcccttgtccgaccgtcagagaatccaatagacgatccgctgcaccgcatgataaacaatgagcgtgtgtttgtgtgtgcgtgagagcgcgcgtgcctgcctgtcgcgcgcgtgtccttgtcgaacacggaacacacaccagcggatggcgccggtcgccgcgctcgcctccccggccggcttctcgatcagccgcggctgtcaaggaggggaaagggcccggccgcgcgtggtggggagcgggaaaagcgccaaaagccacccgcttttgcacggaaccgtggcaagaccacaatgattcctgcggttccgcctcactggccactgcacgccgcgacccgcttttaacttccacgccgccgccgagagctttccaccgccgtcgccgtcggccggtgacgagcgagcgaaagggcctgtgcatgtgcctggctcttgcgccgtcgaccgaacccaggccagacgttcggtctttcttactaagtcgtgtgctcgacgacgctcgcttttcgttggaaaattgaccgtagttggtgggccggccggcaggatatattcaattgtaaatcgagctgttggctggctggggcaggatatattcaattgtaaatcaaattgacgcttagacaacttaataacacattgcggacgtttttaatgtactgaagtcacatccgtttgatacttgtctaaaattggctgatttcgagtgcatctatgcataaaaacaatctaatgacaattattaccaagcatcaatgagatgatgtgtgtgtctatgtgcatgctctagaagcacttaaagatctttagaagaaagcaaagcatttattaatacataacnaatgtccaggtagcccagccgaanttacaatacgcaactgctcataataattcaacaaacccaagtagtacacanatccagaagcaaataagcccatacgtaccaaancctacacaagcagcaacnctcactnncnnngccnngggncnttaaagcacacngnnntgaccanncgatcnnnnngaaacnaactnnnnnatnaanncaancnnaagcnnnnncnnnncnnnnannntnctgntcgnnnnnnnnnnnngnnnnnnnntannanggnnnnnnnnnnnnnnnnnnnnnnnnnnnnnannnnnnnannnnnnnnnnnnnn

SEQ ID NO: 9 - DNA, 505 bp, Artificial Sequence
  106685[1]-010 chromosomal flanking sequence adjacent to an inserted transgene
agacgatccgctgcaccgcatgataaacaatgagcgtgtgtttgtgtgtgcgtgagagcgcgcgtgcctgcctgtcgcgcgcgtgtccttgtcgaacacggaacacacaccagcggatggcgccggtcgccgcgctcgcctccccggccggcttctcgatcagccgcggctgtcaaggaggggaaagggcccggccgcgcgtggtggggagcgggaaaagcgccaaaagccacccgcttttgcacggaaccgtggcaagaccacaatgattcctgcggttccgcctcactggccactgcacgccgcgacccgcttttaacttccacgccgccgccgagagctttccaccgccgtcgccgtcggccggtgacgagcgagcgaaagggcctgtgcatgtgcctggctcttgcgccgtcgaccgaacccaggccagacgttcggtctttcttactaagtcgtgtgctcgacgacgctcgcttttcgttggaaaattgaccgtagttgg

SEQ ID NO: 10 - DNA, 1250 bp, Artificial Sequence
  106685[1]-013 chromosomal flanking sequence, adapter, and cloning vector sequence which was produced by the method
nnnnnnnnnnnnnnncntgctcgagcggccgccagtgtgatggatatctgcagaattcgcccttgtccgaccgtcagagaatccaatagacgatccgctgtaccttcggacacgaacccttcggacaagtcaaaattgttgtatgaagaaacacaaaaagaacgaaggatggaacagagccgaagctgtgcgcaaggaagcttcagcatgatggcaaaaaagaggaaaccgacttaaagaggaaaagactatttagaccccgatagattactatagagttattatcaaaagcaaagggcctggatgtaattttacatggtccgcgacccgtacctataaataggtgaacagtactcccgtactgttcacactgacttggtatttgcttttgcatcacgctcgtacttttaccttctttcaagtcaaaggtatatttgtaacccaacatcattcctattcttctatagcaataaaataaaaatgagttaataataatacataactattcatgttatcttttatattccatgtaatccccttttcattatgatatgtaatgtttatgaaggtatgtccttcataaccttcatccaaaaatcattatatctcaagggaaataatgcttcgaaggacgaaggactttaacatttaacgttttctgtgttgccttgttcttaactcttagcatttgagaacaagtgaccaacaaatcttcttgttgaaaatgaacttttatcttgcaatacacatgaggctattaaccctgttgttaagattgatgtagcaacctcatgtgatgatttgagtcaaggagagcaaactagtctacatgatgaattgaccgaaaaagttgaagtcttgactttagacaaccaaaaattgaagagatgcttgactaatgcaactactataggaanngntgctattgagaaaaatgnnttcaacaatgagttggcagcgcataatgaaaggcttaaaatgaggtcnagaaacttaagnntgaaaatgaacatcttgcacaagtgnnnnaaagttcaacanggcnaatacntccaaatgaattgcttatnacanntcatgaaaaacannnnnnnggtattngnnnnantgctttnnnncaaannannnanntncnnanannannnaancnnannnatannnnnntnnnnnnntnnantgnnnnnnnngnnnnncnnnnntnncannnnacnncnnnnncnttnnctannnnnnnnnnnnanannnnnnnn

SEQ ID NO: 11 - DNA, 1163 bp, Artificial Sequence
  106685[1]-013 chromosomal flanking sequence adjacent to an inserted transgene
agacgatccgctgtaccttcggacacgaacccttcggacaagtcaaaattgttgtatgaagaaacacaaaaagaacgaaggatggaacagagccgaagctgtgcgcaaggaagcttcagcatgatggcaaaaaagaggaaaccgacttaaagaggaaaagactatttagaccccgatagattactatagagttattatcaaaagcaaagggcctggatgtaattttacatggtccgcgacccgtacctataaataggtgaacagtactcccgtactgttcacactgacttggtatttgcttttgcatcacgctcgtacttttaccttctttcaagtcaaaggtatatttgtaacccaacatcattcctattcttctatagcaataaaataaaaatgagttaataataatacataactattcatgttatcttttatattccatgtaatccccttttcattatgatatgtaatgtttatgaaggtatgtccttcataaccttcatccaaaaatcattatatctcaagggaaataatgcttcgaaggacgaaggactttaacatttaacgttttctgtgttgccttgttcttaactcttagcatttgagaacaagtgaccaacaaatcttcttgttgaaaatgaacttttatcttgcaatacacatgaggctattaaccctgttgttaagattgatgtagcaacctcatgtgatgatttgagtcaaggagagcaaactagtctacatgatgaattgaccgaaaaagttgaagtcttgactttagacaaccaaaaattgaagagatgcttgactaatgcaactactataggaanngntgctattgagaaaaatgnnttcaacaatgagttggcagcgcataatgaaaggcttaaaatgaggtcnagaaacttaagnntgaaaatgaacatcttgcacaagtgnnnnaaagttcaacanggcnaatacntccaaatgaattgcttatnacanntcatgaaaaacannnnnnnggtattngnnnnantgctttnnnncaaannannnanntncnnanannannnaancnnannnatannnnnntnnnnnnntnnantgnnnnnnnngnnnnncnnnnntnncannnnacnncnnnnncnttnnctannnnnnnnnnnnanannnnnnnn

SEQ ID NO: 12 - DNA, 1032 bp, Artificial Sequence
  106685[1]-035 chromosomal flanking sequence, adapter, and cloning vector sequence which was produced by the method
nnnnnnnnnnnnnnnnatgctcgagcggccgccagtgtgatggatatctgcagaattcgcccttgtccgaccgtcagagaatccaatagacgatccgctgtgccgtgagggtgaggaggacagaactgacctacattgggtctgtagatgacgacagctgatacgtgtatgattatcttggtgaattggaggcaaagaatttggtgtcacttgttggcatttgtgtcgtgtgcaagttaaaagcaccaactgcatcgaattctttgagagctctgtaccacaaggtggaatggaaaccatatggcttttggttggttgttgtagtgttagaatacatcgctgcccccgatttgttttagggcctgtgggggcggctgtatatagttggaatccatttctggattcaatgggggagcttatttgagaatcaggcaggaagccatgtagtagatttctagccaagagccctgtacttgtttttttttgggtctgagatgtaattgtcatgtcacatatattcttttgatctaaggctgacggaagttgcattgcctatttgctttagctattactagtgcagcctagacatcatgagtttggatcgttctatgactaaaccgtcctactggtttgctgtttgtttttgtggtataagcttcctttagcgatgtattgttgcttaatataaaggtaatgtagagtttagctattggagatagataactaactgttttctccttttgaggatcatagttaaagtcgactttcctacacatctctttctaatgctactgtgctttanaatcccattacatattggtgttttgaaaatacttattgggtatgttcaaatcaaccccnntataaactttttttnaatgttactaccctagccttgttatcatcatttcanacttttgacntgaaaaangcaactcaaaccaacatantttaagtcggtttggttctcngcctatctaaagttagccctatctaagttagttanttaannaaaagaacnanncntnnata

SEQ ID NO: 13 - DNA, 945 bp, Artificial Sequence
  106685[1]-035 chromosomal flanking sequence adjacent to an inserted transgene
agacgatccgctgtgccgtgagggtgaggaggacagaactgacctacattgggtctgtagatgacgacagctgatacgtgtatgattatcttggtgaattggaggcaaagaatttggtgtcacttgttggcatttgtgtcgtgtgcaagttaaaagcaccaactgcatcgaattctttgagagctctgtaccacaaggtggaatggaaaccatatggcttttggttggttgttgtagtgttagaatacatcgctgcccccgatttgttttagggcctgtgggggcggctgtatatagttggaatccatttctggattcaatgggggagcttatttgagaatcaggcaggaagccatgtagtagatttctagccaagagccctgtacttgtttttttttgggtctgagatgtaattgtcatgtcacatatattcttttgatctaaggctgacggaagttgcattgcctatttgctttagctattactagtgcagcctagacatcatgagtttggatcgttctatgactaaaccgtcctactggtttgctgtttgtttttgtggtataagcttcctttagcgatgtattgttgcttaatataaaggtaatgtagagtttagctattggagatagataactaactgttttctccttttgaggatcatagttaaagtcgactttcctacacatctctttctaatgctactgtgctttanaatcccattacatattggtgttttgaaaatacttattgggtatgttcaaatcaaccccnntataaactttttttnaatgttactaccctagccttgttatcatcatttcanacttttgacntgaaaaangcaactcaaaccaacatantttaagtcggtttggttctcngcctatctaaagttagccctatctaagttagttanttaannaaaagaacnanncntnnata